(19) United States
(12) Patent Application Publication
(10) Pub. No.: US 2025/0381059 A1
(43) Pub. Date: Dec. 18, 2025

(54) MENSTRUAL CUP WITH SANDWICH STRUCTURE

(71) Applicant: Shenzhen Shengyou Technology Co., Ltd, Shenzhen (CN)

(72) Inventors: Shaojun Zhang, Shenzhen Guangdong (CN); Liuqing Bei, Jiexi Guangdong (CN)

(73) Assignee: Shenzhen Shengyou Technology Co., Ltd, Shenzhen (CN)

(21) Appl. No.: 18/986,681

(22) Filed: Dec. 18, 2024

(30) Foreign Application Priority Data
Jun. 14, 2024 (CN) 2024213584293

(51) Int. Cl. A61F 5/455

(52) U.S. Cl. CPC A61F 5/4553

(57) ABSTRACT

Disclosed is a menstrual cup with a sandwich structure, including a cup body and a short handle, where the short handle is not communicated with the cup body, the cup body is in an inverted bell shape, a circular opening is formed at a top of the cup body, and a bottom of the cup body is sealed. The cup body further includes an inner wall and an outer wall, a sandwich layer is arranged between the inner wall and the outer wall at an opening of the cup body, and an internal support ring is arranged in the sandwich layer. The cup body is made of a soft material, the outer wall of the cup body is tightly attached to the body of a user, and when the cup body is compressed by the body, the internal support ring supports the cup opening.

### CROSS REFERENCE TO RELATED APPLICATION

This application claims priority to Chinese Patent Application No. 2024213584293, filed on Jun. 14, 2024, which is hereby incorporated by reference in its entirety.

### TECHNICAL FIELD

The present disclosure relates to the technical field of menstrual products, and specifically relates to a menstrual cup with a sandwich structure.

### BACKGROUND

Highly absorbent sanitary pads are the most common menstrual care products used by women during their menstrual periods. The sanitary pads are relatively soft and capable of absorbing a certain amount of liquid, but they are wrapped around vaginas of users and easily rub against their bodies. No matter what soft materials are used, the sanitary pads will bring some discomfort to the users. Moreover, due to easy side leakage and poor breathability, the sanitary pads will make the users feel uncomfortable in hot weather, thereby necessitating frequent replacement and consumption of massive resources. Menstrual cups are also available on the market, which can replace the sanitary pads and be placed in vaginas of users. Such menstrual cups are primarily made of latex or silica gel, and are soft and elastic. A traditional menstrual cup is bell-shaped with a short handle at a bottom. A bell-shaped portion of the traditional menstrual cup is snap-fitted into the vagina of a user to collect menstrual blood from her uterus, and the short handle helps to keep the menstrual cup balanced in the vagina and make it easy to remove the menstrual cup. Such menstrual cups can be cleaned with water for reuse, and therefore, popularization of the menstrual cups is of great significance.

However, menstrual cups of the prior art, due to single hardness, have the follow defects in use.

After a low-hardness menstrual cup is placed in the vagina of a user, its cup opening cannot be bounce-opened, thereby preventing the menstrual cup from functioning effectively, and causing deformation of the cup body when compressed by the user's body, as well as side leakage due to poor fit between the cup body and the user's body. In contrast, a high-hardness menstrual cup can overcome the defects of failure to bounce-open or deformation, but has poor softness of the cup body, thereby resulting in obvious foreign body sensation, discomfort, and even pain from friction with the user's body.

Therefore, a menstrual cup with a sandwich structure is provided to solve the above problems.

### SUMMARY

In order to overcome the defects of a menstrual cup of the prior art including single hardness, failure to bounce-open a cup opening for fitting the body of a user, and easy side leakage, an objective of the present disclosure is to provide a menstrual cup with a sandwich structure.

To achieve the above objective, the present disclosure adopts the following technical solution: a menstrual cup with a sandwich structure, including a cup body and a short handle fixedly connected to a lower end of the cup body, where the short handle is not communicated with the cup body, the cup body is in an inverted bell shape, a circular opening is formed at a top of the cup body, and a bottom of the cup body is sealed; and the cup body further includes an inner wall and an outer wall.

Further, a sandwich layer is arranged between the inner wall and the outer wall at an opening of the cup body.

Further, an internal support ring is arranged in the sandwich layer.

Compared with the prior art, the present disclosure has the following advantages and effective effects:

In the present disclosure, the sandwich layer is arranged between the inner wall and the outer wall at the opening of the cup body, the harder internal support ring is arranged in the sandwich layer, the internal support ring has both the supporting and rebounding functions, and the internal support ring is harder than the cup body, such that the cup opening can rebound quickly after being folded, which overcomes the defect that the menstrual cup cannot rebound after being placed in a vagina; the cup body is made of a soft material, the outer wall of the cup body is tightly attached to the body of a user, and when the cup body is compressed by the body, the internal support ring supports the cup opening, such that the cup opening is tightly attached to the body without deformation or leakage; and the soft material of the cup body enhances comfort, and alleviates foreign object sensation and pain caused by friction with the body.

### BRIEF DESCRIPTION OF THE DRAWINGS

FIG. 1 is a sectional view of a side surface of a menstrual cup with a sandwich structure provided by the present disclosure.

FIG. 2 is an oblique exploded view of a menstrual cup with a sandwich structure provided by the present disclosure.

Reference numerals in the figures: 1. cup body; 2. short handle; 3. inner wall; 4. outer wall; and 5. internal support ring.

### DETAILED DESCRIPTIONS OF THE EMBODIMENTS

In order to make the objectives, technical solutions and advantages of the embodiments of the present disclosure clearer, the technical solutions in the embodiments of the present disclosure will be clearly and completely described below in combination with the accompanying drawings in the embodiments of the present disclosure. Apparently, the embodiments described are merely some rather than all of the embodiments of the present disclosure. Based on the embodiments of the present disclosure, all other embodiments obtained by those of ordinary skill in the art without making creative efforts shall fall within the protection scope of the present disclosure.

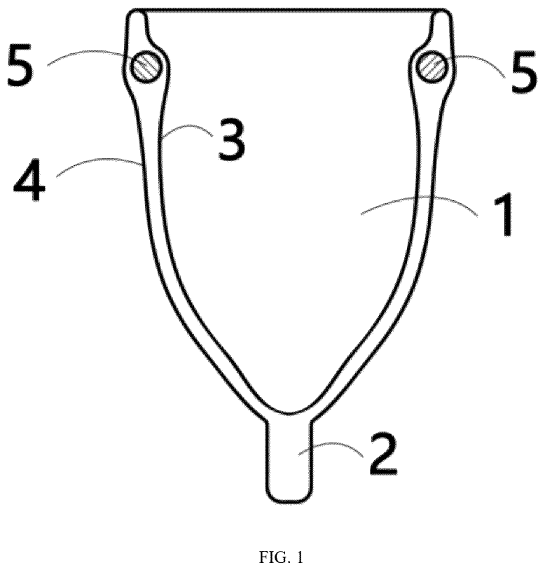

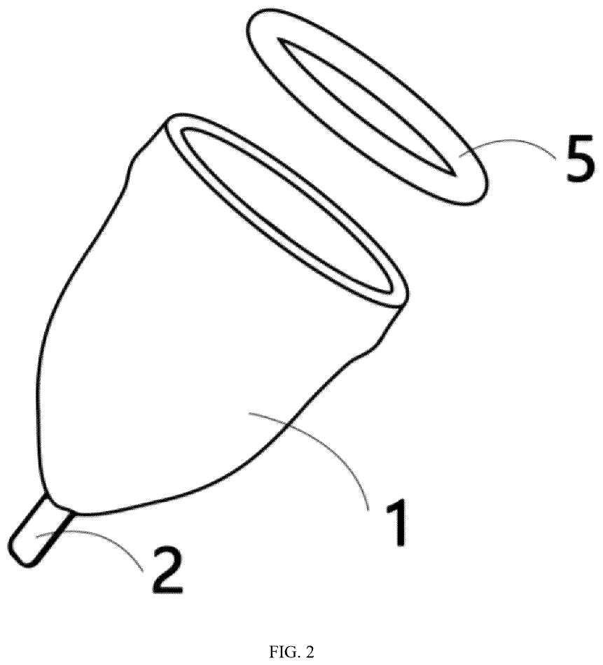

With reference to FIGS. 1-2, the present disclosure provides the following technical solution: a menstrual cup with a sandwich structure, including a cup body 1 and a short handle 2 fixedly connected to a lower end of the cup body 1, where the short handle 2 is not communicated with the cup body 1, the cup body 1 is in an inverted bell shape, a circular opening is formed at a top of the cup body 1, and a bottom of the cup body 1 is sealed. The cup body 1 further includes an inner wall 3 and an outer wall 4.

Specific arrangement and functions of the menstrual cup are described in detail below.

In the implementation solution, a sandwich layer is arranged between the inner wall 3 and the outer wall 4 at the opening of the cup body 1.

Specifically, an internal support ring 5 is arranged in the sandwich layer.

In the implementation solution, the internal support ring 5 has both supporting and rebounding functions, the internal support ring 5 is made of environmentally friendly silica gel or plastic, the cup body 1 is made of softer environmentally friendly silica gel or plastic, and the internal support ring 5 is harder than the cup body 1, such that the cup opening can rebound quickly after being folded.

Working principle: For better use, the sandwich layer is arranged between the inner wall 3 and the outer wall 4 at the opening of the cup body 1, the harder internal support ring 5 is arranged in the sandwich layer, the internal support ring 5 has both the supporting and rebounding functions, and the internal support ring 5 is harder than the cup body 1, such that the cup opening can rebound quickly after being folded, which overcomes the defect that the menstrual cup cannot rebound after being placed in a vagina; the cup body 1 is made of a soft material, the outer wall 4 of the cup body 1 is tightly attached to the body of a user, and when the cup body 1 is compressed by the body, the internal support ring 5 supports the cup opening, such that the cup opening is tightly attached to the body without deformation or leakage; and the soft material of the cup body 1 enhances comfort, and alleviates foreign object sensation and pain caused by friction with the body.

What is claimed is:

1. A menstrual cup with a sandwich structure, comprising a cup body and a short handle fixedly connected to a lower end of the cup body, wherein the short handle is not communicated with the cup body, the cup body is in an inverted bell shape, a circular opening is formed at a top of the cup body, and a bottom of the cup body is sealed; and the cup body further comprises an inner wall and an outer wall.

2. The menstrual cup with a sandwich structure according to claim 1, wherein a sandwich layer is arranged between the inner wall and the outer wall at an opening of the cup body.

3. The menstrual cup with a sandwich structure according to claim 2, wherein an internal support ring is arranged in the sandwich layer.
